(19) United States
(12) Patent Application Publication
(10) Pub. No.: US 2008/0275348 A1
(43) Pub. Date: Nov. 6, 2008

(54) Monitor device and use thereof

(75) Inventors: Michael Catt, Sharnbrook (GB); Ming Li, Sharnbrook (GB); Arthur Maurice Weightman, Bromham (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc.d/b/a Unilever

(21) Appl. No.: 12/150,679

(22) Filed: Apr. 30, 2008

(30) Foreign Application Priority Data
May 1, 2007 (GB) 0708457.7

(51) Int. Cl. A61B 5/11; A61B 5/01; A61B 5/0205

(52) U.S. Cl. 600/483; 600/595; 600/549

(57) ABSTRACT

In an apparatus and method for obtaining an indication of energy expenditure by a mammal during exercise, one or more movement transducers (1) each output a respective movement signal related to physical movement. A frequency analysis (3) is performed on at least one of the movement signals to obtain a frequency analysis result. Classification means (5) determines from the frequency analysis result, what class of physical movement is involved in the exercise. Selection means (7) selects a form of calculation according to a class determined by the classification means. A form of calculation selected by the selection means is applied (9) to at least one of the movement signals so as to obtain the energy expenditure indication.

### FIELD OF THE INVENTION

The present invention relates to a monitor device and its use. More especially, it relates to a monitor apparatus or device for obtaining an indication of the amount of energy expenditure during a given period of exercise. It also relates to a method for obtaining an energy expenditure indication of the aforementioned kind.

As used herein, the term “energy expenditure indication” means a quantitative, semi-quantitative or qualitative indication of the amount of energy expended by a mammal over a period of time. The present invention is primarily useful for obtaining an indication of energy expenditure by a human but may also be used for certain mammalian animals, such as racehorses.

### BACKGROUND OF THE INVENTION

There has been much interest in ways of obtaining an indication of the energy expended during exercise, not only for athletes and other persons engaged in sport, but also for the normal population, i.e. children and adults, including young adults, as well as middle aged and older people.

Energy expenditure by a person arises from muscle and metabolic expenditure. Movement sensing can yield an estimate of muscle derived energy expenditure only. The food calorific requirements are therefore a function of metabolic conversion and utilisation efficiency of body movement. The correlation between velocity and acceleration does not hold when attempting to determine true energy expenditure.

It is already known to derive an estimate of energy expenditure over time, using an accelerometer worn by a subject. A simple algorithm or function is applied to the values output by the accelerometer, in order to derive an indication of the energy expended over time (e.g. see US-A-2004/681039). Since energy is a function of velocity and not acceleration, the values obtained in this way are necessarily approximate but nevertheless, in principle, can give a reasonably accurate result if the function (algorithm) is chosen carefully.

However, using an accelerometer alone has another drawback. It may give similar readings for completely different kinds of activity. For example, an accelerometer worn on the wrist will react to arm movement and could give similar readings, both for a person driving and a person running. Obviously, the energy expended in the latter case is much greater than the former. Therefore, a recent proposal utilises not only the output of an accelerometer but also a measurement related to a physiological parameter such as basal metabolic rate, as disclosed in US-A-2004/0249315.

Further, it is known (US-A-2004/681039) to apply simple algorithms to the output of energy transducers to obtain an approximation of energy expenditure.

‘Existing methods described in the prior art do not address the practical constraints imposed in realising a means of physical activity classification compatible with implementation in a compact device for wearing on the body. These limitations include processing power, memory, power consumption and cost’.

We have now found that greater accuracy in energy expenditure determination can be obtained using a transducer with an output related to movement of the subject, such as an accelerometer, if the type of movement involved in the exercise is first classified. The present invention achieves this classification by performing a frequency analysis on the transducer output. According to the classification obtained (type of exercise being undertaken), a suitable form of calculation can be chosen to convert the transducer output into the energy expenditure indicator.

### DEFINITION OF THE INVENTION

Therefore, a first aspect of the present invention provides an apparatus for obtaining an indication of energy expenditure by a mammal during exercise, the apparatus comprising:
(i) one or more movement transducers, each for outputting a respective movement signal related to physical movement;
(ii) analysis means' for performing an analysis on the at least one of the movement signals to obtain an analysis result;
(iii) classification means for determining from the analysis result, what class of physical movement is involved in the exercise;
(iv) selection means for selecting a form of calculation according to a class determined by the classification means; and
(v) means for applying a form of calculation selected by the selection means to at least one of the movement signals so as to obtain said energy expenditure indication.

A second aspect of the present invention provides a method of obtaining an indication of energy by a mammal during exercise, the method comprising:
(i) obtaining one or more movement signals related to physical movement;
(ii) performing an analysis on the at least one of the movement signals to obtain an analysis result;
(iii) using the analysis result to determine what class of physical movement is involved in the exercise;
(iv) selecting a form of calculation according to the determined physical movement class; and
(v) applying the selected form of calculation to at least one of the movement signals to obtain said energy expenditure indication.

### DETAILED DESCRIPTION OF THE INVENTION

The present invention requires at least one signal related to physical movement to be obtained. This signal may be a single signal related to one kind of physical movement, for example acceleration. The signal is obtained in practice, using an appropriate transducer. For example, one kind of signal related to physical movement is an acceleration signal, which may be obtained from an accelerometer. Miniature accelerometers based on piezoelectric or capacitive devices are commercially available. Another kind of signal related to movement is one related to velocity. Velocity may, for example, be obtained from a portable GPS unit. Yet another form of signal related to physical movement is a count of number of steps taken (foot-floor) which may be obtained as the output from an electronic pedometer.

In the simplest realisation, a single transducer may be employed. For example an accelerometer. Alternatively, a plurality of transducers of the same type or of differing types may be employed. For example, one could be worn on a wrist band and another of the same general type could be worn on the clothing close to the torso. If necessary, another could be attached to an ankle.

In a preferred class of embodiments, at least two transducers are employed in a manner to produce output signals respectively related to movement in different directions, for example along two or three different substantially mutually orthogonal axes.

Optionally, as well as one or more transducers of a given type, one or more other transducers for obtaining a different kind of movement related signal could also be employed, attached to (worn by) the subject. For example, one or more velocity and acceleration transducers may be utilised and/or one or more pedometer-type transducers.

The output or outputs from the movement transducer(s) comprise a signal or signals which are subjected to an analysis to produce a result that can be used to classify the class of activity being undertaken by the subject. In the following description, the singular ‘signal’ and ‘signals’ in the plural can be used interchangeably and where one is expressed, the other may also be assumed, unless the context explicitly forbids.

The analysis result is used to determine the kind of physical movement which is being undertaken in the exercise being monitored. This can be done by comparing the analysis result with a plurality of data sets stored in a library. In most practical realisations, this library will be stored in a computer memory device, such as a semi-conductor memory or disk memory. Preferably, the data sets will have been created by a calibration technique in which the transducer or transducers and analysis means will be used to create data sets from subjects performing predetermined exercises.

Therefore, preferably, the kind of analysis used to create the stored data sets during calibration will be the same as the analysis used to produce the analysis result on actual subjects under investigation, whose energy expenditure is to be estimated or determined.

The stored data sets may be updated and improved by means of an adaptive empirical method, such as using a Kalman filter or a neural network. This may be done from further calibration exercises or using actual data from subjects under investigation. Some different kinds of analysis of the movement signals (and signals used for calibration) will now be explained in more detail.

The simplest kind of analysis which may be used is a frequency analysis, i.e. the analysis of the movement signals comprises an analysis of frequency components of those signals. Any suitable frequency analysis known to those skilled in the art may be employed, for example Fourier analysis or wavelet analysis. The result of such frequency analysis is then utilised to determine the kind of movement being undertaken by the subject, e.g. using a simple look-up table or an algorithm or algorithms. Neural net techniques may also be applied to provide an on-going update of what kind of frequency/amplitude spectrum is most indicative of a given class of activity.

Fourier analysis and wavelet analysis are well known tools and software for performing either is commercially available. Fourier analysis involves representation of a complex waveform as the sum of a number of sinusoidal waves of differing frequency and amplitude. Wavelet analysis and its practical application is described in depth in M. V. Wickerhauser, “Adapted Wavelet Analysis from Theory to Software”, A.K. Peters Ltd., 1994, ISBN 1-56881-041-5.

One preferred way of classifying Fourier analysis data is to map the individual intensities (amplitudes) for the various frequency components and also to map the ratios of chosen significant frequency components. Some daily activities are characterised by characteristic underlying frequencies (walking and running) whilst others are much less so (resting, driving, typing & writing). Frequency analysis is especially suitable for activities with repetitive movement where appropriate frequencies can easily be identified from the amplitude vs. frequency plot for the signal derived from each measurement axis in turn by selection of the prominent signal peaks that change most characteristically on transition from one repetitive movement to another (e.g. walking to running). The maps of frequency amplitude against frequency and of the ratios of the significantly changing peaks are then stored for later reference for new data. When a data set is to be classified, it is compared against the stored maps of intensities and ratios. The type of activity corresponding to the new data set is then identified by selecting the most closely matching amplitudes and ratios by standard error minimisation methods.

However, the most preferred way of analysing the transducer output(s), i.e. the movement signals in order to classify a given type of activity is first to create a map in Cartesian three dimensional vector space for each individual movement (acceleration magnitude and direction expressed in x, y, z co-ordinates). This will result in a surface in the x, y, z space which is characteristic of the particular type of activity giving rise to the data. In the method described here the absolute acceleration values obtained from any specific axis is replaced by consideration of the contribution of each specific axis to the overall resultant acceleration at each time point thus normalising the coordinates of the map and emphasising the angular contribution of the respective axes to the immediate acceleration direction.

When a data set of transducer outputs is obtained from an ‘unknown’ class of activity, a surface in three-dimensional vector surface is generated in the same way. This surface is then compared with each of the surfaces stored in the library to determine the closest match by standard error minimisation methods for powerful processor systems or specific banded thresholds assigned for the movement derived from the reference datasets.

In another class of embodiments, sampled data is subject to data transformation, feature extraction and combination, followed by incorporation into a combined feature vector (dimensionality reduction). The last step involves implementation of a classification model. The combined feature vector typically involves calculation or estimation of power (energy for time unit), contour profiling and generation of a rotation profile. The classification model may be a BayesNet, decision tree or a so-called support vector machine.

The two methods described below allow the classification of physical movement from a triaxial accelerometer attached to a body through analysis of the changes in the cosine of resultant acceleration vector relative to the specific (defined) axes of the device. This data may be combined with the absolute resultant or the integral of this measure over time to provide more detailed information about the movement of the body and the energy expenditure incurred. The underlying distributions of these cosines of one axis against a second or third axis are characteristic for many common daily activities. By calculation of particular patterns and comparison to known reference patterns acquired from a population or from the individual, periods of movement and rest can be classified. Special consideration is given here to practical methods compatible with execution by a low-powered processing unit classifying activities in real time and embedded with the accelerometer in a compact unit for unobtrusive mounting on the body. Such methods can be compatible with extended periods of monitoring (hours, days or weeks) between battery recharge (replacement) and in the storage on the device in a compact form of the activity intensity (integrated acceleration for a time period (epoch)) and a characteristic identifier of the dominant activity for the epoch (e.g. walking, typing, shopping etc). Such data may then be recovered by wireless (or other) link for consideration of lifestyle or clinical significance at appropriate, convenient intervals. The second method extends the first to illustrate how sub-classification or additional contextual information of relevance may be derived from the cosines. In this case, the simple Haar wavelet transform permits resolution of specific characteristics hidden within the signal (in this example to distinguish walking on the flat from an incline uphill or downhill).

The relevant activity type corresponding to the closest match with library surfaces is then used as the basis of the energy expenditure computation.

Classes (categories) of exercise which may be discriminated in this way include driving, walking, running, swimming, climbing and various household activities such as gardening, vacuum cleaning, bed making, ironing and the like.

The invention may be used to monitor a subject over a time which may include more than one type of exercise, perhaps as well as period of low activity or rest.

When the kind of movement has been classified, according to the classification determined, an appropriate form of calculation is selected to treat the signal or signals related to the physical movements, i.e. from the movement transducers to convert them into an indication of energy expenditure. Again, this calculation may take the form of use of a simple look-up table or application of one or more algorithms.

The energy expenditure indicator may for example be a numerical value, or a simple classification such as “low”, “moderate” or “high” energy expenditure and may be displayed by any suitable means such as an alphanumeric display (e.g. of LED or liquid crystal type), an analogue meter or a “traffic light” system (e.g. green for “low”, amber for “moderate” and red for “high” energy expenditure).

A more sophisticated evolution of this basic system can also utilise the output of one or more secondary transducers which produce signals related to one or more physiological parameters such as heart rate, peripheral pulse rate or skin temperature transducers. All of these are available commercially. One or more of any one or more types of these secondary transducers may be employed. The output of such secondary transducer(s) may be employed directly or be subjected to further signal processing, before being used in the classification, which in any event is also utilising the frequency analysis of the movement signals, in order to better obtain a classification of the type of exercise movement being undertaken.

In practical realisations, any transducer or transducers may be carried in any suitable form for wearing by the subject, e.g. in wrist bands or modules to be attached to the clothing. They may all be housed in a single unit or in separate units. Such unit or units may contain all or part of the other means for carrying out the frequency analysis, classification and final calculation to obtain the energy expenditure indicator. The latter functions may be carried out by suitable hard-wired circuitry and/or software in a microprocessor based apparatus. Any or all of these parts may also be housed in an apparatus having another primary function, such as a wrist watch, a mobile phone, portable music player or personal digital assistant (PDA). Any such module or modules may also be provided with means for inputting, e.g. keypad, one or more parameters which may also be employed in the calculation to obtain the energy expenditure indicator, such as body weight and age of subject.

The present invention will now be described in more detail by way of the following description of preferred embodiments, and with reference to the accompanying drawings in which:—

### BRIEF DESCRIPTION OF THE DRAWINGS

FIG. 1 shows a block diagram explaining the operation of a monitor according to a first embodiment of the present invention;

FIG. 2 shows a plot of average scalar acceleration value against estimated MET values for different categories of activity;

FIG. 3 shows histograms of the distribution of angular contributions to the resultant along one axis plotted against the contribution made from a second axis;

FIG. 4 shows the Haar wavelet;

FIG. 5 shows a complete map of a continuous wave transform for one volunteer;

FIG. 6 shows an analogous comparison to that shown in FIG. 5, for another volunteer;

FIG. 7 shows comparative data for the same volunteer as in FIG. 6 and also for another volunteer;

FIG. 8 shows analysis of the x²r² (cosine²) data using the Haar wavelet of another volunteer ‘mc’ walking outdoors;

FIG. 9 shows the analysis of the y²/r² (cosine²) data using the Haar wavelet of volunteer ‘mc’ walking outdoors for the same volunteer as in FIG. 8; and

FIG. 10 shows a plot of the amplitude of the Haar transform coefficient for the cases depicted in FIG. 9.

### DESCRIPTION OF PREFERRED EMBODIMENTS

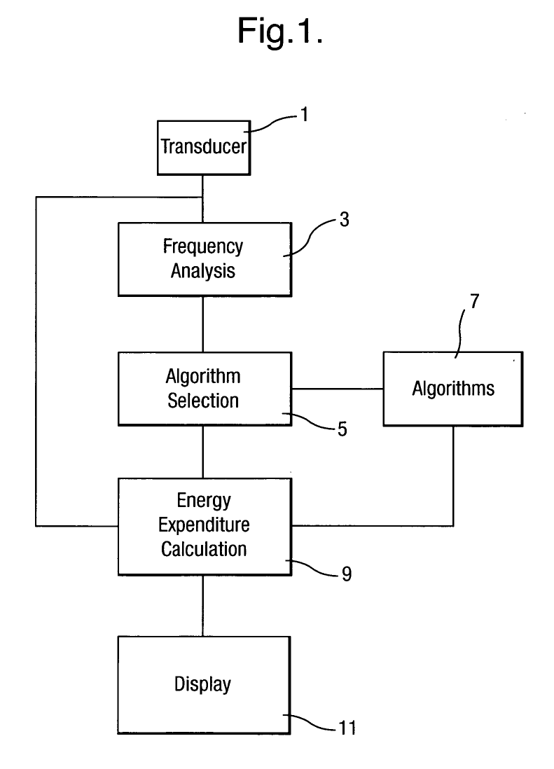

A block diagram explaining the operation of a monitor according to a first embodiment of the present invention, is depicted in FIG. 1. In this embodiment, a transducer 1 which is a three axis (x, y, z) accelerometer, produces outputs which are processed by an electronic processing unit. Further details of the accelerometer are given below. In this processing unit, the output of the transducer is subjected to a frequency analysis by the wavelet technique using commercial software, as indicated by numeral 3. The result of the frequency analysis is used in an algorithm selection step 5 in which an appropriate one of algorithms stored in an algorithm store 7 is selected according to the result of the frequency analysis. The appropriate algorithm is applied in an energy expenditure calculation 9 to the output of the transducer 1. An indication of energy expenditure over a predetermined time is thus obtained and is visible to a user on display 11.

Experimental evaluation of the above-described system has proved that it is capable of distinguishing between the activities of driving, walking, running and domestic housework and of generating a separate energy expenditure evaluation for each.

A preferred embodiment will now be described, based on the vector surface calibration and analysis technique.

### (i) Equipment and Methods

In this embodiment, subjects used for calibration and for evaluation each wore a triaxial accelerometer on the dominant wrist in a manner similar to a wrist watch. The STMicroelectronics LIS3LV02DQ triaxial accelerometer generates a 12-bit digital output proportional to acceleration on each of three orthogonal axes denoted x, y and z. An accelerometer with analogue outputs could equally be used with the signals being digitised using a separate analogue-to-digital converter. The output data rate (ODR) of the accelerometer was set to 160 Hz which sets its digital filter cut-off frequency to 40 Hz (ODR/4). The data was read by a PIC18LF2520-I/ML microcontroller where it was formatted and time stamped prior to wireless transmission via a CSR BlueCore BC358239A-INN-E4 chip and associated antenna either to a Bluetooth enabled PC or hand held computer with appropriate data reception software. Software in a PC computer (MATLAB) is configured to perform calibration and analysis as will be described in more detail below to produce estimates of energy expenditure.

### (ii) Calibration

Subjects wearing these transducers were each instructed to undertake one of the following activities whilst wearing the transducer, namely, standing still with arms relaxed and hanging normally at the side of the torso, walking on a treadmill at 4 kmph, 5 kmph and 6 kmph, running on a treadmill at 8 kmph and 10 kmph, sitting typing at a desk on a PC keyboard, sitting writing at a desk on a sheet of A4 paper transcribing predefined paragraphs onto A4 paper. Signals received by the computer, in digital form, consisted of instantaneous accelerations in each of the three axes recorded at 160 samples per second with each observation time stamped to allow identification of data sequences associated with specific activities.

These signals were processed electronically to create, in electronic form, both the square of the instantaneous scalar resultant (rₜ²) acceleration for each time point and the square of the cosine of each axis vector acceleration (xₜ²/rₜ², yₜ²/rₜ², zₜ²/rₜ²).

i.e. where

Cos²(⊖^{x}ₜ)=xₜ²/rₜ²

Cos²(⊖^{y}ₜ)=yₜ²/rₜ²

Cos²(⊖^{z}ₜ)=zₜ²/rₜ²

where theta is the angle between the designated axis and the resultant for the instantaneous measurement.

Such vectors can be mapped against each other in three dimensional (square of vectors only) or four dimensional space (square of vectors and scalar resultant) to illustrate the specific characteristics of movements associated with particular activities.

The coordinates of the resultant surface were stored in a library, for each calibration subject, together with a designation of which class of activity running, stair climbing etc, which gave rise to that particular surface.

The resultant electronically stored ‘surfaces’ thus, consisted of an array of multi-dimensional variables (xₜ²/rₜ², yₜ²/rₜ², zₜ²/rₜ² and rₜ²). Additional variables were calculated from these data. The high sample rate of 160 Hz and circuit design captures frequency contributions above those normally characteristic of human movement. Filtering of the variable rₜ² allows the respective contributions of different frequencies to the resultant to be emphasised or de-emphasised. In this case high frequency contributions can be suppressed using a simple exponential smoothing filter of the form:

Sₜ=αyₜ+(1−α)Sₜ₋₁

where the smoothing constant (a) may be ⅛ and the starting value (S₀) may be chosen from the first signal in the sequence (i.e. S₀=y₀) or the average of the signal sequence to be characterised with other initial estimates also usable. Such filters have a low processing overhead and are compatible with low-cost, low-power processor realisation.

An exponential filter with a smoothing constant of ⅛ as applied here to this data yields a high frequency 3 db cut-off of 3.4 Hz. The data as originally sampled is filtered by the electronic arrangement with a high frequency 3 db cut-off of 40 Hz. Physiological movement signals will mostly lie below 10 Hz, with major activities predominantly contributing below 5 Hz. The choice of 3.4 Hz is selected here to show the adequacy of such a break point, but 1.6 Hz (smoothing constant of 1/16) is also very suitable. Similarly, the computation of only the squares and not the signed vectors eliminates the need for square-root calculation and allows reversal of axes when wearing the device to give added flexibility for the wearer in consumer applications.

For clinical or higher-value consumer applications, where the characterisations can be performed off-line, or more powerful on-board processing can be implemented, the signed vectors may be calculated explicitly and used in association with the device mounted in specific orientation to identify more detail classification and asymmetry of movement. In the simple example here, the histograms are constructed from the square of the contributions (e.g. x²/r²). Acceleration on any axis may be positive or negatively vectored against an axis and so the explicit contribution of x/r may therefore be signed. The histograms will thus map between −1 and +1 rather than 0 and 1. Asymmetry of movement will be manifest by characteristic changes in the histogram functions in these explicit contributions and reference thresholds for contributions established by similar methods to those described here to characterise both normal and abnormal movement.

Data presented in this specification was calculated using only the first signal of the sequence as might be supposed from basic engineering texts describing the method.

To facilitate characterisation of the activity in a chosen period, in this case over one minute, the sum of the absolute gradients of the unfiltered square of the resultant and the sum of the absolute gradients of the low frequency filtered square of resultants is calculated and the ratio of the two numbers conveys information concerning the type of activity. i.e.

Cusum(r²)=sum(abs(gradient(r²)))

Ratio=Cusum(r²)/Cusum(smoothed r²)
where smoothed r² represents the exponentially smoothed values of r²

The gradient function simply calculates the step difference between the current and previous value which is a straight forward subtraction for any low-cost processor. The square root function is very processor intensive so the r2 values are used throughout. The characteristic ratio is stored in a simple look-up table referenced to the type of activity within the final processor.

For example, the data presented later in this specification show that running and walking give rise to a ratio of between 1.5 and 1.9 typically whilst typing yields a ratio of 3.1 to 3.7 and writing from 3.8 to 4.5. Standing still yields ratios between 2.4 and 3.0 typically. This ratio therefore allows differentiation between bipedal activity and the sedentary activities of writing and typing typical of general office work. The high frequency signal component may also arise from sources other than the immediate human activity (e.g. transmitted from the motion of a motor vehicle). Such movements can generate high cumulative sums/average of rₜ² values or similar indices but not be associated with a high level of specific energy expenditure by the person. This high ratio arising allows the differentiation of the human activity from the transmitted movement typical of common human-machine interactions.

The squares of the cosines are used to establish characteristic indices of particular activities using processing methods compatible with low-power, low-cost processing capability for real-time evaluation.

Within the selected epoch (again one minute in this case), histogram functions are constructed such that map the cumulative sum of each of the other two axes against selected bands of values on the third axis. For illustrative purposes, within the epoch of interest where xₜ²/rₜ²>=0 and <0.1 the cumulative sum of all corresponding yₜ²/rₜ² and zₜ²/rₜ². Similarly cumulative sums are calculated for >=0.1 to <0.2, and so forth to >=0.9 <=1 to yield ten ‘bins’ on each axis. The movement associated with activity generates characteristic distributions within these histograms such that simple rules can be devised.

When considering the cumulative sums of zₜ²/rₜ² against the individual histogram bins along xₜ²/rₜ² for instance, standing and walking are characterised by a maximum in bin 1 (i.e. the cumulative sum of all zₜ²/rₜ² is highest where xₜ²/rₜ² is >=0 and <0.1) with reducing levels in bins 2, 3, 4 and very low cumulative sums in the higher bins. The two activities can be distinguished by simple thresholds with the cumulative sum in bin 1 for standing typically <50 and for walking above 50 but below 150. Running at 10 kmph again shows a decline in values from bin 1 to bin 4, with substantial but lower values in higher bins (<50) but the bin 1 value exceeds 200.

Thus, each of the three activities can be simple discriminated one from the other. Further, typing peaks in bin 2 or bin 3 (with values above 2000) with little signal in the high bins (6, 7, 8, 9, 10). Writing peaks at higher bins (6, 7) at values exceeding 800 in this arrangement. Therefore, it is clear that all of the activities can be differentiated one from the other by such simple rules. Similar rules can be devised and applied to other histograms in this sequence with reasonable satisfaction but zₜ²/rₜ² versus xₜ²/rₜ² most easily discriminates for these activities. Thus for any set of activities and population of individuals with allowance for the specific filtering and signal properties of the triaxial accelerometer can a method be devised with this approach. Depending on the range and subtlety of distinction it is clear that it is not always necessary to calculate cumulative values of the two complementary axes for each of the three axes or to calculate for all bins. Moreover, the number of bins can be modified according to the sophistication desired and the processing capability available.

In the embodiments described below, a workable discriminator is established calculating just zₜ²/rₜ² versus xₜ²/rₜ² and calculating only cumulative sums only for those bins that provide the discrimination (minimally, 1, 3, 6 with preferably 8) to minimise processing overhead. The threshold values described above can again be stored in a simple look up table such that the logical rules as explained can be applied to any new data epoch for real-time classification.

A more sophisticated approach is to compute the typical characteristic histograms from a range of humans participating in the selected activities and to compare observed histograms for ‘goodness of fit’. This can reasonably be calculated by calculating the residual sum of the squares of the differences between individual bins for each activity type and classifying the activity to that for which the minimum difference is observed.

Once activities have been classified according to type, it is possible to generate an estimate of energy expenditure for that activity. Energy expenditure for specific activities have been collated by Barbara Ainsworth (Med Sci Sports Exerc. 2000 September; 32(9 Suppl):S498-504. ‘Compendium of physical activities: an update of activity codes and MET intensities (Ainsworth B E, Haskell W L, Whitt M C, Irwin M L, Swartz A M, Strath S J, O'Brien W L, Bassett D R Jr, Schmitz K H, Emplaincourt P O, Jacobs D R Jr, Leon A S). The MET is defined as the rate at which an adult human consumes energy per hour and is approximately 1 kcal per kg body weight per hour. Figures derived from indirect calorimetry for specific reference activities are stored in the computer and have been used to calculate energy expenditure in practice.

Alternatively, energy expenditures for particular activities are measured in a representative sample of individuals using any of various known methods (Am. J. Clin. Nutr. 1999 May; 69(5):920-6. ‘Equations for predicting the energy requirements of healthy adults aged 18-81 y’. Vinken A G, Bathalon G P, Sawaya A L, Dalial G E, Tucker K L, Roberts S B.) or by combined heart rate and accelerometry (J Appl Physiol. 2004 January; 96(1):343-51. ‘Branched equation modeling of simultaneous accelerometry and heart rate monitoring improves estimate of directly measured physical activity energy expenditure’. Brage S, Brage N, Franks P W, Ekelund U, Wong M Y, Andersen L B, Froberg K, Wareham N J.) either in the laboratory or in free living individuals.

Typically such estimates will generate estimates of energy expenditure for walking or running at different speeds. A common approach to interpreting accelerometer data has been to associate the cumulative sum of the absolute value of rₜ for an epoch (e.g. typically 1, 2, 5 or 10 minutes) or some related measure (mean resultant over time for instance) and to map this against energy expenditure.

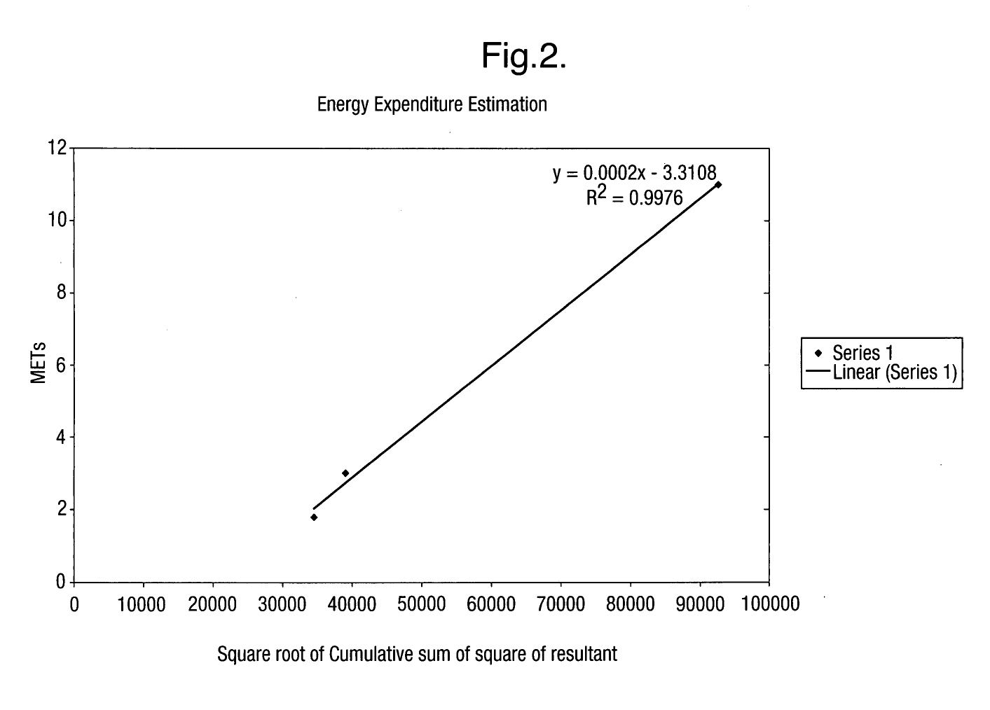

The relationship for average square root of all four volunteers of their Cusum (rₜ²) for the estimated MET values for standing, walking and running as detailed in Table 1 (below) is illustrated in FIG. 2. Whilst such relationships work acceptably well for the range of normal bipedal activities they are confounded by other normal daily activities. The use of the classifiers described here allows an initial assignment to a class of activity and an estimated energy expenditure in accordance with the energy expenditures associated with the range of intensities recorded from the accelerometer output for the type of activity.

(iii) Measurements

After calibration of the system as described above, individuals whose energy expenditure is to be determined, were instructed to wear the transducer(s) of the aforementioned kind for a prescribed period of monitoring.

During this evaluation period, the signals from the transducer(s) were transferred to the computer by the same means as described above for the calibration process. These signals consisted of a data stream which was evaluated every minute to determine the class of activity being undertaken. A time burst of data again comprising 160 samples per second from each axis for a period of one minute (xₜ²/rₜ², yₜ²/rₜ², zₜ²/rₜ² and rₜ²). Again, this variable set was stored temporarily as an array of multi-dimensional variables of the aforementioned kind. This data set may be considered to be an “analysis result” in the sense used in the claims of this specification. On completion of the one minute epoch, the Cusum (rₜ²) using the unfiltered square of resultants and the Cusum (smoothed rₜ²) using the low frequency-filtered square of resultants was calculated and the ratio of the two numbers used as previously described to differentiate the type of activity. Similarly differentiation of the activities was demonstrated by calculation of the cos² histogram and application of the discrimination rules by reference to the look-up table of thresholds. Thus a cumulative sum of 65×10⁶ equates to walking at 4 kmph. Estimates of energy expenditure

The results for four individuals are shown in Table I:

TABLE I
Comparison of One minute Integrals for a variety of activities for four
individuals and the matching MET table estimates of energy expenditure.

| | J′ | A′ | R′ | E′ | METS |
|---|---|---|---|---|---|
| 10 kmph run | 1313.5 | 1372.7 | 1257.7 | 1283.7 | 11 |
| LF-R | 760.4 | 708.6 | 730.1 | 756.8 | |
| ratio | 1.7 | 1.9 | 1.7 | 1.7 | |
| 4 kmph walk | 35.4 | 54.5 | 70.6 | 51.3 | 3 |
| LF-R | 23.2 | 28.9 | 47.9 | 33.4 | |
| ratio | 1.5 | 1.9 | 1.5 | 1.5 | |
| Standing | 9.6 | 2.5 | 9.1 | 6.7 | 1.8 |
| LF-R | 4.0 | 0.8 | 3.8 | 2.7 | |
| ratio | 2.4 | 3.0 | 2.4 | 2.5 | |
| Typing | 52.0 | 70.6 | 27.5 | 51.0 | 1.5 |
| LF-R | 15.0 | 19.4 | 8.4 | 16.7 | |
| ratio | 3.5 | 3.7 | 3.3 | 3.1 | |
| Writing | 63.6 | 60.6 | 25.0 | 55.3 | 1.8 |
| LF-R | 16.2 | 13.8 | 5.5 | 14.5 | |
| ratio | 3.9 | 4.4 | 4.5 | 3.8 | |

One minute Cusum (rₜ²) (× 10⁶) for a variety of activities for four individuals and the matching MET table estimates of energy expenditure.

In Table I, the Cusum (rₜ²) is shown for each of four individuals (J, A, R and E) against the specific activities (10 kmph run, 4 kmph walk, standing still, typing and writing) and underneath the Cusum (smoothed rₜ²) using the first value of the epoch as the starting value and an alpha of ⅛. The ratio of the unfiltered to filtered is then compared against the reference values generated from the calibration data for the look-up table. Although both typing and writing produce Cusum (rₜ²) higher than that obtained for walking, which by normal means of interpreting accelerometer data would suggest higher activity levels in these two activities, the ratio value of >3.1 for typing and >3.8 for writing clearly differentiates these activity types from the >1.5<1.9 indicative of normal walking.

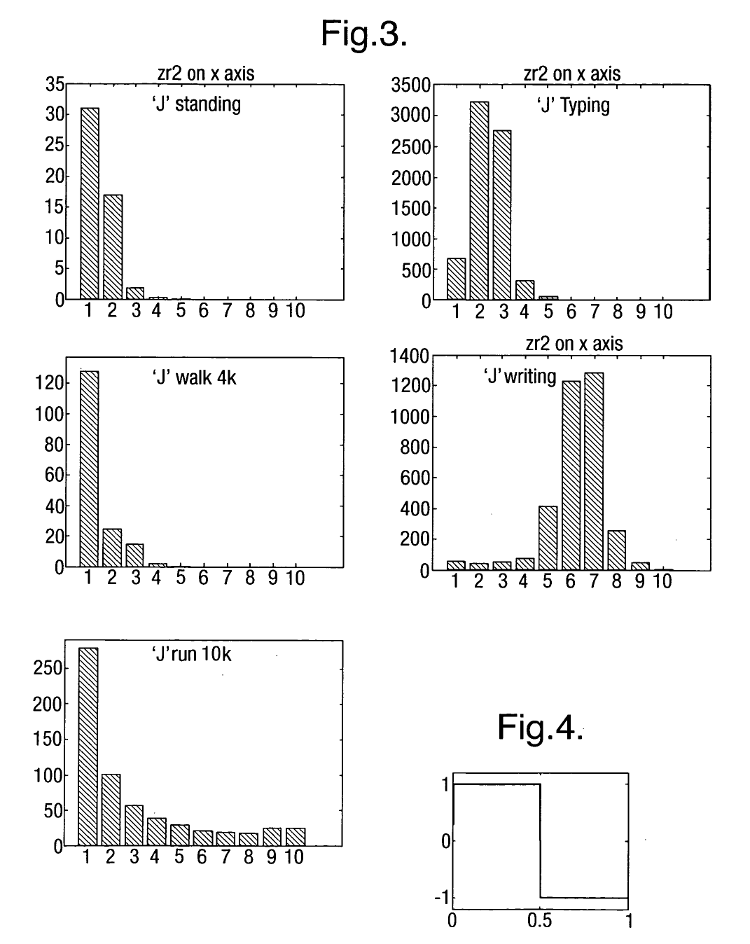

A histogram map for ‘J’ further is shown in FIG. 3. This illustrates the additional means of differentiating between the activities. Such plots show how strongly the first axis contributes to the resultant when the second axis is contributing within a certain defined range towards the resultant. For ease of computation the contribution of the y axis to the resultant is represented as y²/r², the x axis as x²/r² and the z axis as z²/r². The histograms are generated by measuring the respective acceleration signals on each axis (in this case 160 times per second) and calculating a resultant acceleration r²=x²+y²+z² over a defined period (in this case one minute). The contribution of any axis to the square of the resultant is therefore the square of the acceleration measured on that axis. Thus the range of values for any axis can lie between 0 and 1 for the squares. If this axis is then divided into segments, the cumulative sum of the first axis squares associated by time point for all of the second axis contributions in a specified range will thus generate a histogram

According to the rules devised in the calibration, the maximum in bin 1 for zₜ²/rₜ² versus xₜ²/rₜ² for standing, walking and running, with Typing showing a maximum in bin 2 and writing in bin 7 clearly differentiates the activity types selected for this assessment. The magnitude of the bin 1 for standing, walking and running further discriminates within the bipedal activity.

Similarly, the approach of classifying the activity according to the minimal difference provides a more sophisticated approach suitable for more powerful processing systems.

### (iv) Energy Expenditure Calculation

For a given determination of the class of exercise being undertaken, until the next evaluation and classification is undertaken, it was assumed that the exercise being undertaken was that corresponding to the classification of the stored data set from the library according to the rule developed above. According to which class of activity is thereby identified, the measurement data from the transducer was then processed to give a calculation of energy expenditure.

For example, if the type of exercise was identified as “running”, the data from the transducer(s) is processed as follows: —

First the set of base variables is constructed (xₜ²/rₜ², yₜ²/rₜ², zₜ²/rₜ² and rₜ²) and evaluated by the means described above to confirm that the activity is indeed likely to be running. The Cusum (rₜ²) for the epoch is evaluated and compared to a look-up table of values for running that relate this measure directly to energy expenditure. The closest estimate is selected. A Cusum (rₜ²) of 1.3×10⁹ counts in this example equates to running at 10 kmph which is estimated from laboratory studies to be 11 METS.

If the class of activity was “Typing” the data from the transducer is input to the following equations to yield the energy expenditure:—

First the set of base variables is constructed (xₜ²/rₜ², yₜ²/rₜ², zₜ²/rₜ² and rₜ²) and evaluated by the means described above to confirm that the activity is indeed classified as typing. In this case, typing is a form of sedentary occupational work with a laboratory estimated energy expenditure of 1.8 METS.

If the class of activity was “Standing” the data from the transducer is input to the following equations to yield the energy expenditure:—

First the set of base variables is constructed (xₜ²/rₜ², yₜ²/rₜ², zₜ²/rₜ² and rₜ²) and evaluated by the means described above to confirm that the activity is indeed classified as standing. In this case, standing has a laboratory estimated energy expenditure of 1.2 METS.

In a second embodiment, instead of the variable set analysis technique, a wavelet analysis approach was used.

The basic protocol for calibration and measurement which was used for the vector surface technique above, was also employed here. However, the process used, for calibration, activity classification energy expenditure estimation and correlation between measurement data and library data were as follows:—

The commercial accelerometer device used for this application is autocalibrated against gravity for this application. Again the individual contributions to the square of resultant are calculated for each observation for the selected axis so normalising the data between zero and one. The method requires certain parameters to be stored to characterise the movement type, including the scale of the wavelet to be applied to the data, the basic wavelet form (in this case, the Haar wavelet), the specific thresholds to distinguish the mean intensity of variation in the continuous wavelet transform coefficient as identified below and the tolerance range for acceptable time periods between successive maxima and minima.

Whilst the above method (method 1) allows discrimination of a representative collection of physical activities, further discrimination may require more advanced techniques. The following describes an approach to resolve changes in incline experienced by a volunteer wearing the triaxial accelerometer described above walking either on a treadmill or ‘free-living’.

The patterns of daily activity recorded by an accelerometer attached to a human body are typically ‘non-stationary’. The continuous wavelet transform is a technique frequently applied to the analysis of such signals since the advent of the modern digital computer and the work of Stephane Mallat (A Wavelet Tour of Signal Processing ISBN: 0-12-4666 06, Academic Press, 1999).

U.S. Pat. No. 6,571,193 describes how time-frequency analysis by Fourier or wavelet methods may be used to retrospectively classify different motions such as walking or running from data acquired from an accelerometer attached to the hip of an individual.

The subtle changes in signal arising from a simple change in gradient on a treadmill for example are not readily resolved by the Fourier transform through consideration of the frequencies arising from footfall frequency alone without consideration of the dynamic angular changes associated with such a transition. In the method disclosed herein, we have adopted an improved approach is to analyse the characteristic angular vector changes associated with particular movements as introduced in the method above by wavelet methods as follows:

As before the base variables are constructed (xₜ²/rₜ², yₜ²/rₜ², zₜ²/rₜ² and rₜ²). A continuous wavelet transform (W) is then calculated using the formula:

$${W}_{ψ} s ( α , τ ) = \frac{1}{\sqrt{α}} {\int }_{- ∞}^{+ ∞} s ( t ) ψ ( \frac{τ - t}{α} ) d t$$

Where t is time, alpha is the scaling factor, tau is the wavelet time displacement, s is the signal and psi is the wavelet. Thus the weight (W) for the signal (s) is obtained for each wavelet scaling and displacement by integrating the product of the signal and wavelet response values at all time points in the series and adjusting the result by the inverse of the root of the scaling factor. The first wavelet to be described was the Haar wavelet that consists of a single square wave cycle, as shown in FIG. 4.

This simple wavelet may easily be constructed and scaled for computation in a low cost microprocessor system.

For speed of computation a look-up table can be constructed for the known set of scaling factors applied in the analysis to give the square-root for the final correlation but in practice, the square may be equally usefully utilised. Moreover, only those scalings and displacements need be considered that provide adequate discrimination of the cases required.

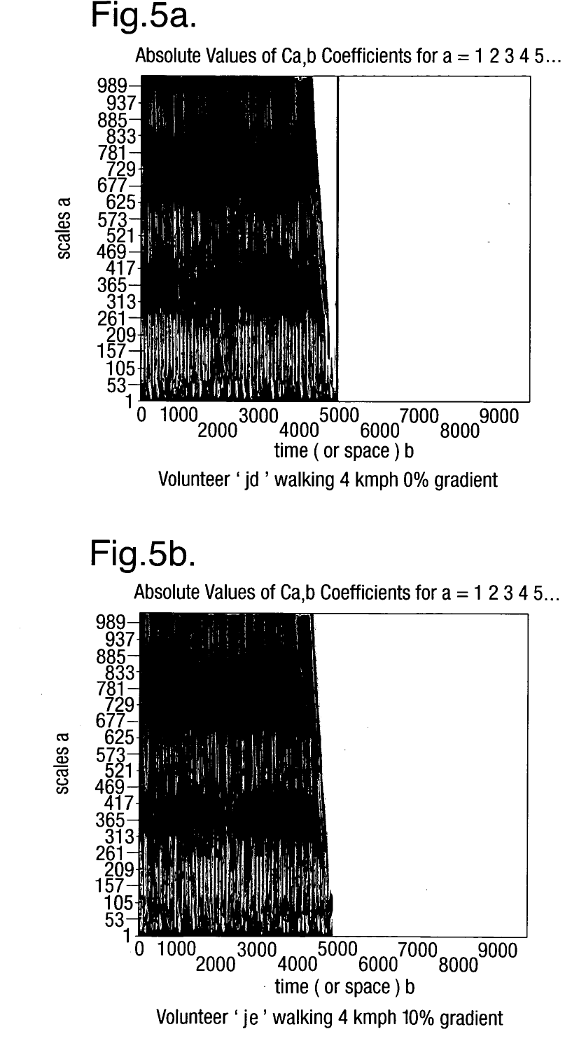

A complete map of the continuous wave transform for volunteer ‘jd’ at 0% gradient calculated on xₜ²/rₜ², whilst the volunteer was walking at 4 kmph on a treadmill is shown in FIG. 5.

The equivalent map for the same volunteer on increasing the gradient to 10% is shown in the right hand half of FIG. 5.

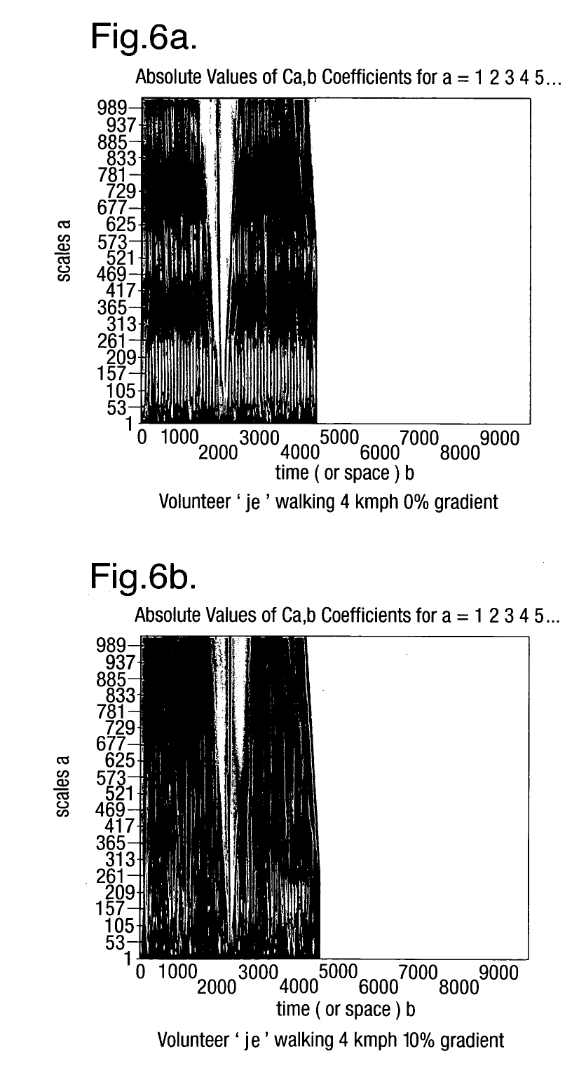

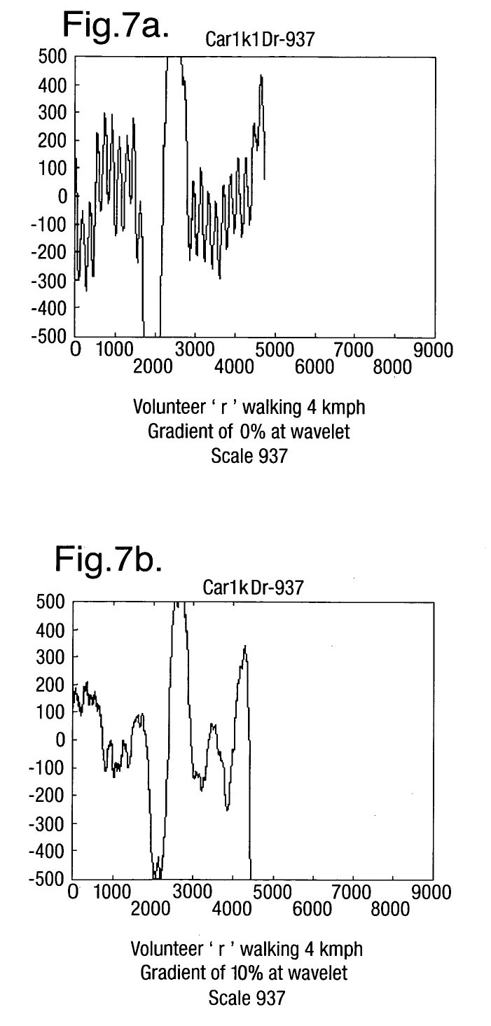

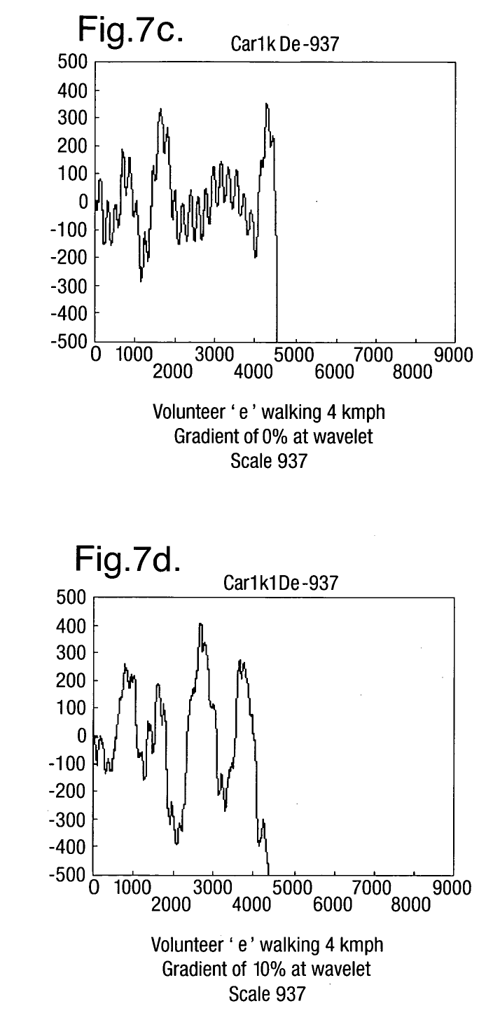

A further comparison for volunteer ‘e’ is shown in FIG. 6.

Comparison of the wavelet coefficients in a specific region of interest highlights characteristic differences in the behaviour of the coefficients between the two cases: In each case, there is a higher density of variation in the 0% gradient compared to the 10% gradient cases with the subjects walking in a normal and relaxed style.

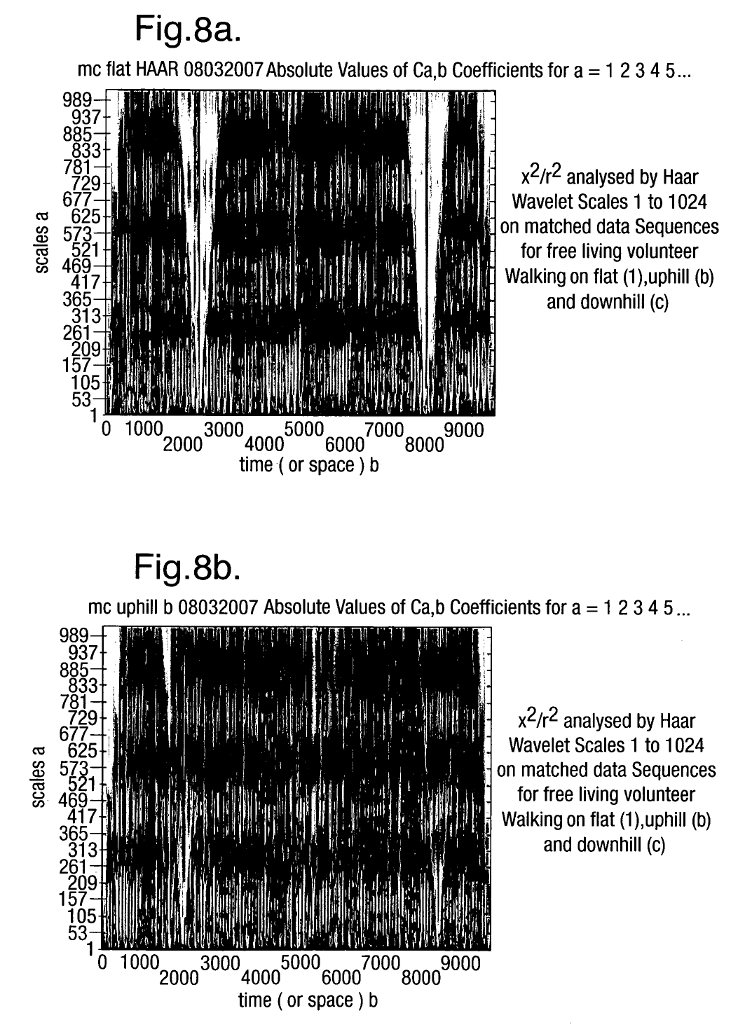

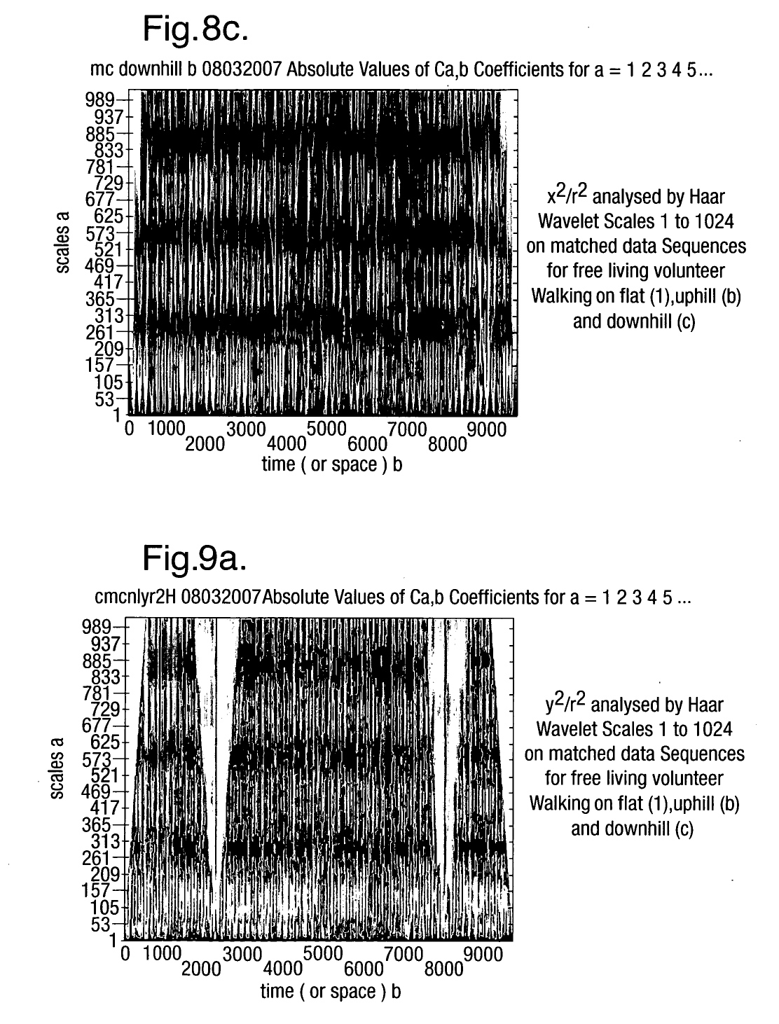

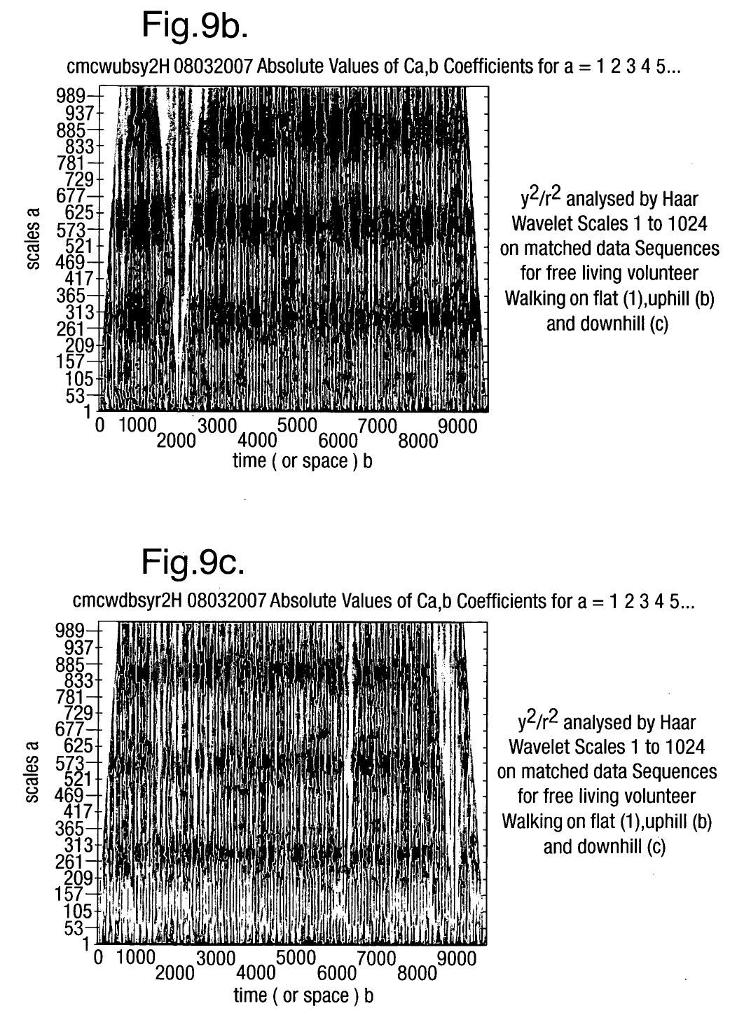

A similar representation of the data collected from another volunteer ‘mc’ walking outdoors is shown in FIGS. 8 and 9. The outdoor and treadmill plots demonstrate similar characteristics in the transition from level to gradient walking.

In this case of the ‘mc’ data outdoors, the x²/r² (cosine²) was been analysed using the Haar wavelet over a scale of 1 to 1024 over 9000 observations (data sampling rate: 160 observations per second on each of the three axes), as depicted in FIG. 8.

It is clear from the aforementioned data that there is an increased variation along the time axis across the scales on the transition from downhill to flat and from flat to uphill.

Analysing the y²/r² cosine sequence using the Haar wavelet also yields distinctive differences, with similar transitions from downhill to flat and then again from flat to downhill, as shown in FIG. 9.

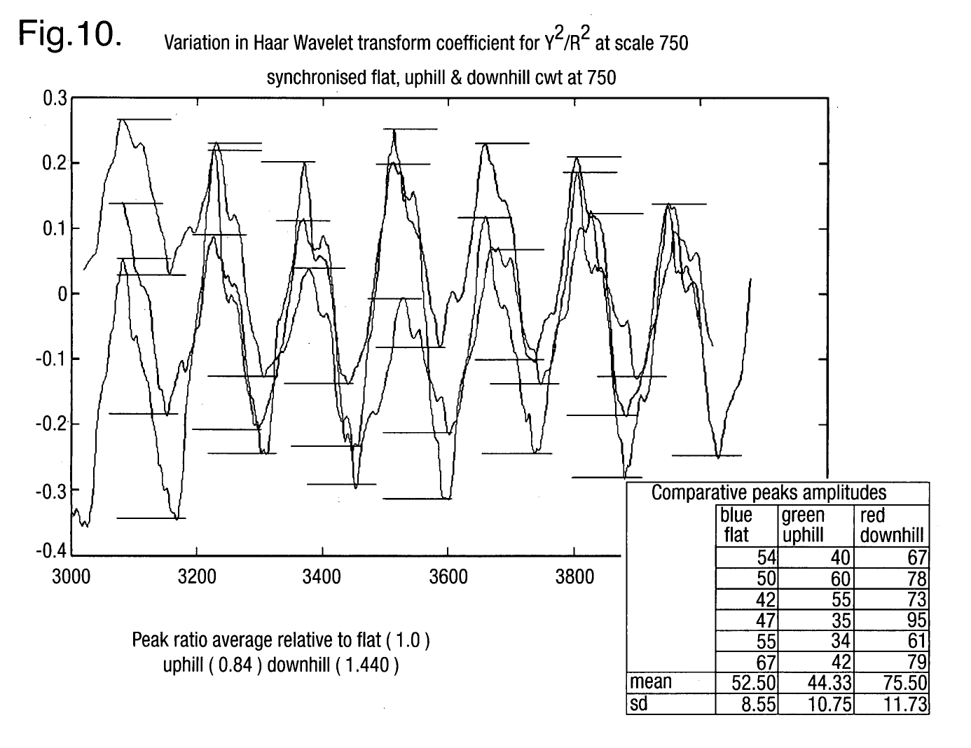

To illustrate this distinctive difference more clearly, FIG. 10 shows the amplitude of the Haar transform coefficient between data points 3000 and 4000 (again at 160 samples per second) for each of the three cases shown in FIG. 9.

The height from successive maximum to successive minimum on an approximately one second interval is measured in arbitrary units and the ratios calculated. The maximum to minimum ratio of the coefficient averages 1.44× that of the flat walking example, whilst the uphill ratio reduces to 0.84. This ratio is dependent on the angle of the incline.

It is clear therefore that there is sufficient difference between the cases that the entire wavelet transform over all scales need not be calculated. In the above case, selecting a scale of 750 and calculating the average height between successive maxima and minima of the Haar wavelet transform of the y²/r² (i.e. cosine² of y axis contribution to resultant) at approximately one second intervals, that is at a frequency that reflects the essential rhythm of the foot placement on walking is sufficient to discriminate the three cases for an epoch length of only six seconds (1000/160 samples). Clearly once the ‘flat’ walking condition has been characterised, a ratio of higher than one indicates downhill progress and less than one indicates uphill progress for that period. Moreover this need only be calculated for a characteristic axis (preferably ‘y’ but ‘x’ may also be used. The ‘z’ axis is much less effective).

Simple algorithms to detect such maxima and minima within acceptable time windows can readily be coded for execution by a microprocessor. For example maxima and minima can be located from either the first or preferably the second differential. If the first differential is used then the zero crossing points may be localised and simple tests for consistency with an expected time base (foot fall frequency) applied.

Such information can then be combined with the general class of the physical activity (and potentially also with the measured intensity of the resultant or individual signals) to not only classify the activity but generate improved estimates of the level of energy expenditure and determine the duration of specific activities undertaken by an individual within a population during waking and resting periods.

Thus from the Ainsworth tables, walking at 3.5 mph on the level has an estimated energy expenditure of 3.3 METS whereas walking uphill at the same speed has an estimated energy expenditure of 6.0 METS. Similarly walking downhill at 2.5 mph is estimated as an energy expenditure of 2.8 METS compared to 3.0 METS on level ground. The footfall frequency is readily derived by measuring the time elapsed between successive impacts (manifested as maxima in the acceleration profile) on (preferably) the y-axis and this can be used to estimate walking speed. Alternatively, the square root of the Cusum (rₜ²) may be used as a basic index as shown in FIG. 2 with correction for specific context. Clearly, individual characterisation and calibration of the method will improve the accuracy of the estimates. Such characterisations may be achieved by requesting the individual to execute specific tasks (such as walking on the flat for one minute in a suitably chosen location). The present device also incorporates wireless communication to facilitate the recovery of specific data for remote analysis and also to allow the recording of specific calibration values within the device via a PC, pocket PC, mobile phone or other wireless communication device. Such calibration values may be transmitted to the device in response to the analysis of specific calibration activities either conducted within the device or remotely (which has the advantage of more rigorous data checking than can reasonably be implemented in a compact, wrist worn device).

It is common for triaxial devices to record an integral measure of acceleration for a selected epoch (period of time). Using the above methods it is possible to assign each such integral an additional flag indicative of the activity class and context (e.g. sitting, typing, writing, walking at x kmph, uphill/downhill, running at y kmph, shopping, cleaning, ironing, etc, etc). Such additional information is not only of benefit for estimating energy expenditure patterns but also for analysing both everyday and clinically relevant activity patterns. Such analyses for example can distinguish gait anomalies, levels of tremor which can provide useful assessments of the effects of medication or behaviour change strategies for improved health or functional performance.

In the light of the described embodiments, modifications of those embodiments, as well as other embodiments, all within the scope of the present invention as defined by the appended claims, will now become apparent to persons skilled in the art.

1. An apparatus for obtaining an indication of energy expenditure by a mammal during exercise, the apparatus comprising:
(i) one or more moement transducers, each for outputting a respective movement signal related to physical movement;
(ii) analysis means for performing an analysis on at least one of the movement signals to obtain an analysis result;
(iii) classification means for determining from the analysis result, what class of physical movement is involved in the exercise;
(iv) selection means for selecting a form of calculation according to a class determined by the classification means; and
(v) means for applying a form of calculation selected by the selection means to at least one of the movement signals so as to obtain said energy expenditure indication.

2. An apparatus according to claim 1, wherein the analysis means is adapted to perform an analysis of the movement signals which comprises an analysis of frequency components.

3. An apparatus according to claim 2 wherein the analysis means is: adapted to perform an analysis of frequency components which comprises Fourier analysis.

4. An apparatus according to claim 2 wherein the analysis means is: adapted to perform an analysis of frequency components which comprises wavelet analysis.

5. An apparatus according to claim 1, wherein the analysis means is adapted to perform an analysis of the movement signals which comprises mapping of movement vectors to create a vector surface.

6. An apparatus according to claim 1, wherein the classification means is adapted to determine the class of physical movement from the analysis result by comparison of the analysis result with members of a library of stored data sets each indication of a respective different class of analysis result to determine which stored data set best matches the analysis result.

7. An apparatus according to claim 6, further comprising a memory for storing the stored data sets and calibration means for creating the stored data sets from calibration results obtained from the analysis means.

8. An apparatus according to claim 7 wherein the calibration means is adapted to update the stored data sets by means of an adaptive empirical method.

9. An apparatus according to claim 8, wherein the adaptive empirical method uses a Kalman filter or a neural network.

10. An apparatus according to claim 1, wherein said one or more transducers is or are, selected from any of accelerometers, velocity transducers and pedometers.

11. An apparatus according to claim 1, comprising at least two of said transducers arranged to produce a respective movement signal related to physical movement in respective different directions.

12. An apparatus according to claim 1, further comprising one or more secondary transducers, each for outputting a respective secondary indication signal, related to respective one or more physiological parameters.

13. An apparatus according to claim 12, wherein said classification means is arranged also to utilise said one or more secondary indication signals and/or to utilise respective signals derived from said one or more secondary indication signals, in order to determine said class of physical movement.

14. An apparatus according to claim 12, wherein said one or more secondary transducers are selected from heart rate transducers, peripheral pulse transducers and skin temperature transducers.

15. A method of obtaining an indication of energy expenditure by a mammal during exercise, the method comprising:
(i) obtaining one or more movement signals related to physical movement;
(ii) performing an analysis on at least one of the movement signals to obtain an analysis result;
(iii) using the analysis result to determine what class of physical movement is involved in the exercise;
(iv) selecting a form of calculation according to the determined physical movement class; and
(v) applying the selected form of calculation to at least one of the movement signals to obtain said energy indication.

16. A method according to claim 15, wherein the analysis of the movement signals comprises an analysis of frequency components.

17. A method according to claim 16, wherein the analysis of frequency components comprises Fourier analysis.

18. A method according to claim 16, wherein the analysis of frequency components comprises wavelet analysis.

19. A method according to claim 15, wherein the analysis of the movement signals comprises mapping of movement vectors to create a vector surface.

20. A method according to claim 1, wherein the class of physical movement is determined from the analysis result by comparison of the analysis result with members of a library of stored data sets each indicative of a respective different class of analysis result to determine which stored data set best matches the analysis result.

21. A method according to claim 20, wherein the stored data sets are created from calibration results obtained from the analysis means.

22. A method according to claim 21, wherein the stored data sets are updated by means of an adaptive empirical method.

23. A method according to claim 22, wherein the adaptive empirical method uses a Kalman filter or a neural network.

24. A method according to claim 1, wherein said one or more movement signals is or are, selected from any of signals related to acceleration, velocity and number of steps taken.

25. A method according to claim 1, wherein at least two movement signals are obtained, respectively related to physical movement in different directions.

26. A method according to claim 1, further comprising obtaining one or more secondary indication signals related to respective one or more physiological parameters.

27. A method according to claim 26, wherein said selection of a form of calculation according to the predetermined physical movement class also utilises said one or more secondary indication signals and/or utilises respective signals derived from said one or more secondary indication signals.

28. A method according to claim 1, wherein said one or more secondary indication signals are related to respective physiological parameters selected from heart rate, peripheral pulse and skin temperature.
